(19) United States
(12) Patent Application Publication
(10) Pub. No.: US 2024/0120068 A1
(43) Pub. Date: Apr. 11, 2024

(54) SYSTEMS AND METHODS FOR IDENTIFYING DEPRESSION IN A DIALYSIS PATIENT

(71) Applicant: Fresenius Medical Care Holdings Inc., Waltham, MA (US)

(72) Inventors: David Yuds, Hudson, WI (US); Jun Yi, New York, NY (US)

(21) Appl. No.: 18/483,390

(22) Filed: Oct. 9, 2023

Related U.S. Application Data
(60) Provisional application No. 63/414,781, filed on Oct. 10, 2022.

(51) Int. Cl. G16H 20/70; G16H 40/67

(52) U.S. Cl. CPC G16H 20/70; G16H 40/67

(57) ABSTRACT

Detailed herein are systems, methods, and devices configured for detecting symptoms of depression in a home dialysis patient. These systems, methods, and device may be embodied in a home dialysis system. The home dialysis system may be configured to carry out a home dialysis treatment on a patient. During the home dialysis treatment, the home dialysis system may be configured to detect one or more symptoms of depression and to generate a medical alert based on the detected one or more symptoms.

### CROSS REFERENCE TO RELATED APPLICATIONS

The present application claims the benefit of and priority to U.S. Provisional Patent Application Ser. No. 63/414,781, filed Oct. 10, 2022, the disclosure of which is incorporated herein by reference.

### FIELD

The disclosure generally relates to treating kidney disease, and more particularly to systems and methods for identifying symptoms of depression in a dialysis patient.

### BACKGROUND

For many patients, home dialysis provides several benefits over in-center dialysis, including improved health outcomes and reduced recovery time. However, efficacious treatment requires proper and consistent usage of the dialysis system by the home patient. Any impediment to consistent use can be a setback to recovery resulting in the need for the patient to seek costly intervention and remedial treatment.

One such impediment is depression, which by some measures may affect as much as 40% of dialysis patients. An individual suffering from depression may fail to use home dialysis equipment correctly or consistently. Home treatment often involves fewer clinical visits and less direct contact with medical professionals, which may form barriers to diagnosing depression, which leads to depression in home dialysis patients often going undetected.

It is with respect to these and other considerations that the present improvements may be useful.

### SUMMARY

This Summary is provided to introduce a selection of concepts in a simplified form that are further described below in the Detailed Description. This Summary is not intended to necessarily identify only key features or essential features of the present disclosure. The present disclosure may include the following various aspects and embodiments:
According to an exemplary embodiment of the present disclosure, a system for detecting symptoms of depression in a home dialysis patient may include a home dialysis system. The home dialysis system may be configured to carry out a home dialysis treatment on a patient. During the home dialysis treatment, the home dialysis system may be configured to detect one or more symptoms of depression. The home dialysis system generates a medical alert based on the detected one or more symptoms.
According to an exemplary embodiment of the present disclosure, a method for detecting symptoms of depression during home dialysis may include carrying out a home dialysis treatment on a patient. The method may further include, during the home dialysis treatment, detecting one or more symptoms of depression. The method may further include generating a medical alert based on the detected one or more symptoms.
According to an exemplary embodiment of the present disclosure, a non-transitory computer-readable medium storing a program executable by a controller detecting symptoms of depression during home dialysis may include carrying out a home dialysis treatment on a patient; detecting one or more symptoms of depression during the home dialysis treatment; and generating a medical alert based on the detected symptoms.
In various of the foregoing and other embodiments of the present disclosure:
   A symptom of the one or more symptoms of depression may be a sleep behavior.
   The home dialysis treatment may occur at least partially while the patient is asleep.
   The method may further comprise identifying a time window associated with the sleep behavior and scheduling the home dialysis treatment to occur during the identified time window.
   The home dialysis treatment may be a peritoneal dialysis treatment including a dwell phase.
   The home dialysis treatment may be scheduled so that the dwell phase occurs during the identified treatment window.
   The data is detected by a medical sensor integrated within the home dialysis system.
   The medical sensor is a pulse oximeter.
   The medical sensor is an electrocardiogram.
   The medical sensor is a photoplethysmography device or an optical heart rate monitor.
   The medical sensor is an ultrasonic sensor.

Further features and aspects are described in additional detail below with reference to the appended Figures.

### BRIEF DESCRIPTION OF THE DRAWINGS

By way of example, embodiments of the disclosed methods and devices will now be described, with reference to the accompanying drawings, in which:

FIG. 1 is a diagram illustrating an exemplary embodiment of systems for use in identifying depression symptoms in a dialysis patient in accordance with the present disclosure;

FIG. 2 is a diagram illustrating an exemplary embodiment of a controller for a home dialysis unit in accordance with the present disclosure;

FIG. 3 is a flowchart illustrating an exemplary embodiment of a method for identifying depression symptoms in a dialysis patient in accordance with the present disclosure; and

FIGS. 4A-4B illustrate an exemplary embodiment of a dialysis machine in a dialysis system configured in accordance with the present disclosure.

### DETAILED DESCRIPTION

Although the systems and methods described herein are related to treatment of renal diseases such as end-stage renal disease (ESRD) and chronic kidney disease (CKD), it is understood that similar systems and methods may be implemented in environments other than treatment of renal diseases and/or with patients other than renal disease patients. For example, systems and methods in accordance with the present disclosure may be utilized to provide a similar methodology for identifying symptoms of depression during a treatment other than dialysis.

Dialysis machines are known for use in the treatment of renal disease. The two principal dialysis methods are hemodialysis (HD) and peritoneal dialysis (PD). During hemodialysis, the patient's blood is passed through a dialyzer of a hemodialysis machine while also passing dialysate through the dialyzer. A semi-permeable membrane in the dialyzer separates the blood from the dialysate within the dialyzer and allows diffusion and osmosis exchanges to take place between the dialysate and the blood stream. During peritoneal dialysis, the patient's peritoneal cavity is periodically infused with dialysate or dialysis solution. The membranous lining of the patient's peritoneum acts as a natural semi-permeable membrane that allows diffusion and osmosis exchanges to take place between the solution and the blood stream. Automated peritoneal dialysis machines, called PD cyclers, are designed to control the entire peritoneal dialysis process so that it can be performed at home, usually overnight, without clinical staff in attendance.

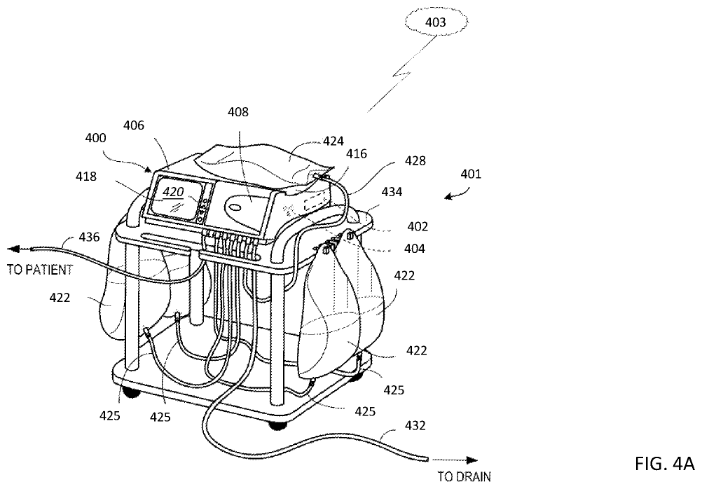

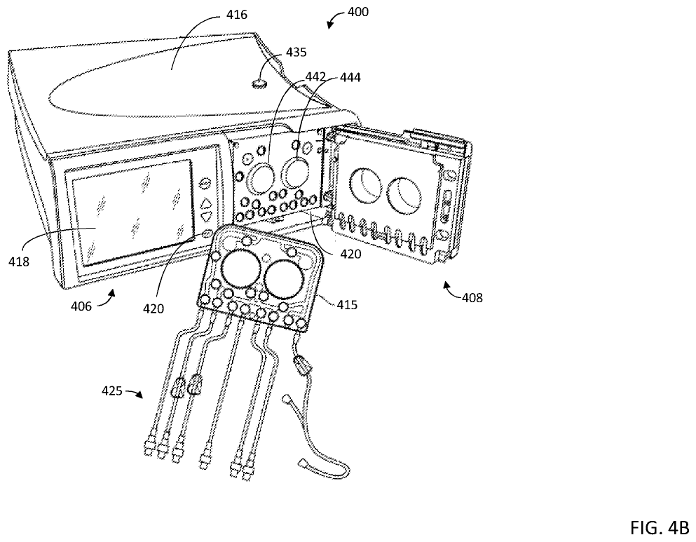

An exemplary embodiment in accordance with the present disclosure may include system 100 for use in treating progression of kidney disease, e.g., End-Stage Renal Disease (ESRD) and/or Chronic Kidney Disease (CKD). Patients with CKD and/or ESRD are patients undergoing long-term care for kidney disease, e.g., by dialysis treatments. For example, some patients may receive dialysis treatments by peritoneal dialysis as described above (see also FIGS. 4A-4B).

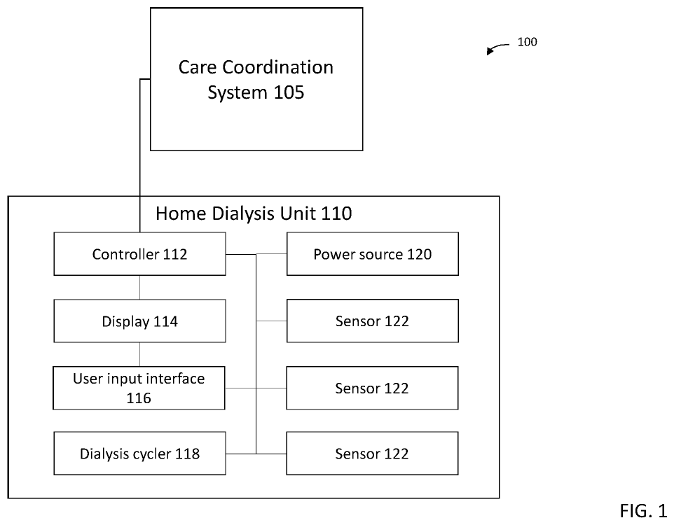

As shown in FIG. 1, the system 100 comprises a care coordination system 105 and a home dialysis unit 110. The care coordination system 105 is in communication with the home dialysis unit 110 as well as with other medical professionals and systems responsible for the patient's care. The care coordination system 105 may allow medical professionals to monitor home dialysis treatment over time and to communicate alterations to the dialysis treatment parameters and schedule to the home dialysis unit 110 as necessary.

The home dialysis unit 110 includes a controller 112, a display 114, a user input interface 116, a dialysis cycler 118, a power source 120, and several sensors 122. In practice the number of sensors 122 can be one or more. Here, three sensors are shown for clarity of presentation. The controller 112 is in communication with each of the other components of the home dialysis unit 110 to both send and receive signals as required for the operation of the unit 110.

The display 114 may be any local output device known in the art for communicating with each user of the home dialysis unit 110, including any patient and/or operator of the unit, such as a medical professional, attendant, or caregiver. The user input interface 116 may be any input mechanism by which a user of the home dialysis unit 110, whether a patient and/or operator, can input data, and in some implementations may be integrated with the display 114 (such as when the user interface 116 include capacitive touch sensors superimposed over the display 114). The display 114 and/or user input interface 116 may be removable in whole or in part from the home dialysis unit 110 to allow for remote control. In some implementations, a user's mobile device may allow for communication with the home dialysis unit 110 to carry out some or all the functions of the display 114 and/or user interface 116.

The dialysis cycler 118 includes the pumps, filters, chambers, valves, fluid lines, and other components necessary to carry out dialysis on the patient in a home environment. An example of a dialysis cycler is shown in FIGS. 4A-B and described therewith.

The power source 120 provides the motive energy to allow the other components of the home dialysis unit 110 to function. In some implementations, different components of the unit 110 may operate on separate or redundant power sources. A portable power supply, such as a battery or generator, may be provided. The power source 120 may include adaptors to residential voltage and current settings. Care should be taken to ensure that regulatory guidelines regarding the supply of voltage to medical devices are followed.

A variety of sensors 122 are included in the home dialysis unit 110. Some sensors 122 are necessary for carrying out the home dialysis treatment itself, such as fluid temperature and pressure sensors associated with the dialysis cycler 118. Certain sensors 122 are provided for monitoring the patient's status.

In different embodiments of the present invention, any or all the following sensors may be included:
Electrical sensors. These include electrocardiograms, electroencephalograms, heart rate monitors, and other sensors for detecting patient vitals by sensing electrical activity and/or changes in electrical resistivity.
Optical sensors. These include photoplethysmograms, optical heart rate monitors, pulse oximeters, and other sensors for detecting patient vitals and gathering data by measuring the refraction and/or diffusion of light through the patient and/or through dialysis fluids.
Sonic sensors. These include ultrasounds and other sensors for gathering data by measuring the interaction of sound waves with the patient and/or dialysis fluids.
Movement sensors. These include mechanical and capacitive devices such as accelerometers for measuring unexpected machine and/or patient movement.

Each of the sensors 122 may be integrated into the home dialysis unit 110 or may be added to the system as an independent component. In some implementations, preparing a patient for dialysis treatment may include deploying one or more sensors on the patient. For example, an electrical sensor may be attached to the patient's skin, an optical sensor may be attached to the patient's extremity, a sonic sensor may be deployed along one or more fluid lines, and a movement sensor may be fastened to the patient's head or arm.

Where a sensor 122 is integrated into the home dialysis unit 110, the sensor 122 may be configured to be deployed in conjunction with attaching components of the dialysis cycler 118 to the patient for treatment. For example, a fluid line that is connected to a patient feed line may also include an electrical line for one or more sensors 122. The sensors 122 may be attached to a patient near where the feed line is deployed.

Sensors 122 may have independent displays, but some sensors may be configured to only provide signals to the controller 112. For example, a pulse oximeter sensor for placement on a patient's digit during a dialysis treatment may not include the display of a standalone pulse oximeter but may instead transmit data, raw and/or preprocessed, to the controller 112 for recording, analysis, and/or display. The display 114 on the unit 110 may, in some configurations, provide the output of the pulse oximeter and other sensors 122 when requested by a user.

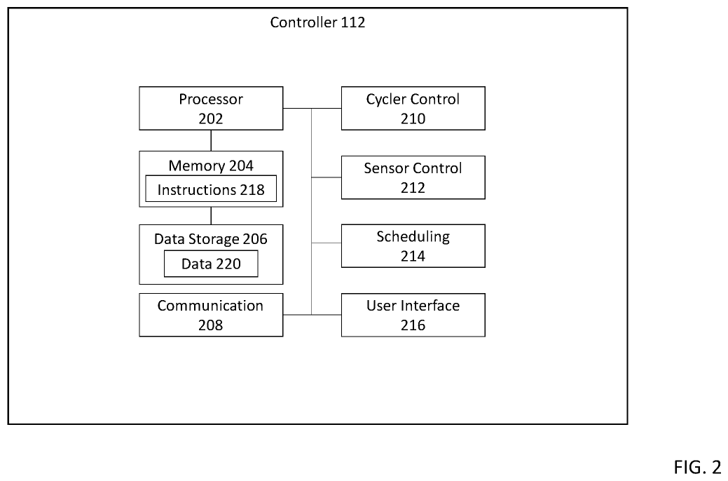

As shown in FIG. 2, the controller 112 is a computing system that includes a processor 202, memory 204, and data storage 206. The controller 112 includes modules associated with each of the functions carried out by the home dialysis unit. In some implementations, the modules described herein with respect to the controller 112 may operate locally within the body of the home dialysis unit, but it will be recognized that some or all the operations may instead be carried out remotely, using remote cloud-based architecture or otherwise.

The controller 112 includes a communications module 208 which may be in communication with a care coordination system through the internet or by other means. The communications module 208 may be used to access remote patient data and may receive instructions for scheduling and configuring patient treatment. Accessed data and stored configuration information may be stored as data 220 in data storage 206 until accessed by the controller 112. In some embodiments, notifications intended for the patient or for a home caregiver may be provided to the home unit by means of the communications module 208.

The controller 112 includes a cycler control module 210 which regulates the functions of the dialysis cycler 118 within the home dialysis unit 110. In some implementations, the cycler control module 210 may include means for sensing the presence or absence of components necessary for carrying out dialysis by means of the dialysis cycler 118, such as fluid bags and/or filtration cartridges. Logic associated with the cycler control module 210 may limit the use of the dialysis cycler 118 if the proper components are not sensed.

A sensor control module 212 governs control of one or more sensors associated with the home dialysis unit 110. In some implementations, the sensor control module 212 may receive data from the sensors and determine whether to store the data 220 within local data storage 220, whether to send the data via the communication module 208 for further analysis, and/or whether to generate an alert via the user interface module 216 involving the data. The sensor control module 212 may ensure that a sensor is properly configured and deployed before data is collected. In some implementations, the absence of properly deployed sensors may cause the controller 112 to postpone or abort a patient dialysis treatment until the sensor problem is identified and remedied.

While described as a single module, it will be understood that, when multiple sensors are deployed with the home dialysis unit, there may be multiple sensor control modules 212 each assigned to different sensors. The amount and type of data collected from different sensors may be different, and each may be independently configured by the controller 112 or by instructions received from a care coordination server or other source.

A scheduling module 214 is used to manage the timing of treatment in accordance with received medical requirements and other constraints provided to the controller 112. These constraints may include a patient sleep cycle, as it is preferred that certain dialysis procedures occur when the patient is asleep.

Modifications to a treatment schedule may occur when certain sensor data of the patient needs to be collected. For example, one potential metric examined for signs of depression is a patient's pulse while asleep during certain times of the night. The scheduling module 214 may schedule the dialysis treatment to occur during the relevant times so that the sensors may collect the required data.

Where the treatment includes multiple phases, and a phase in which the patient is more likely to be asleep, the time of treatment may be adjusted to assure that the appropriate phase occurs during the times that sleep data is to be collected. For example, cyclic peritoneal dialysis includes a “dwell” phase in which cycler pump activity is minimized as dialysis fluid remains within the patient. As patients are encouraged to sleep during these phases, the scheduling module 214 may schedule the treatment such that “dwell” phases are predicted to occur during the key times.

The controller 112 further includes a user interface module 216 to receive input from, and send output to, patients and other users of the home dialysis unit 110. The user interface module 216 may control the display 114 and user input interface 116 of the unit 110. The module 216 may display alerts and messages. In some implementations, users may be able to adjust settings of the home dialysis unit 110, including treatment settings within the scope of what is medically permitted.

It is noted, memory 204 can comprise instructions 218 that are executable by processor 202 and which when executed cause the controller 112 to carry out functions corresponding to components within controller 112. For example, memory 204 can comprise instructions for a scheduling routine, which when executed by processor 202 cause controller to implement functions described for scheduling module 214. As another example, memory 204 can comprise instructions for generating and receiving interactions with a user interface, which when executed by processor 202 cause controller to implement functions described for user interface 216.

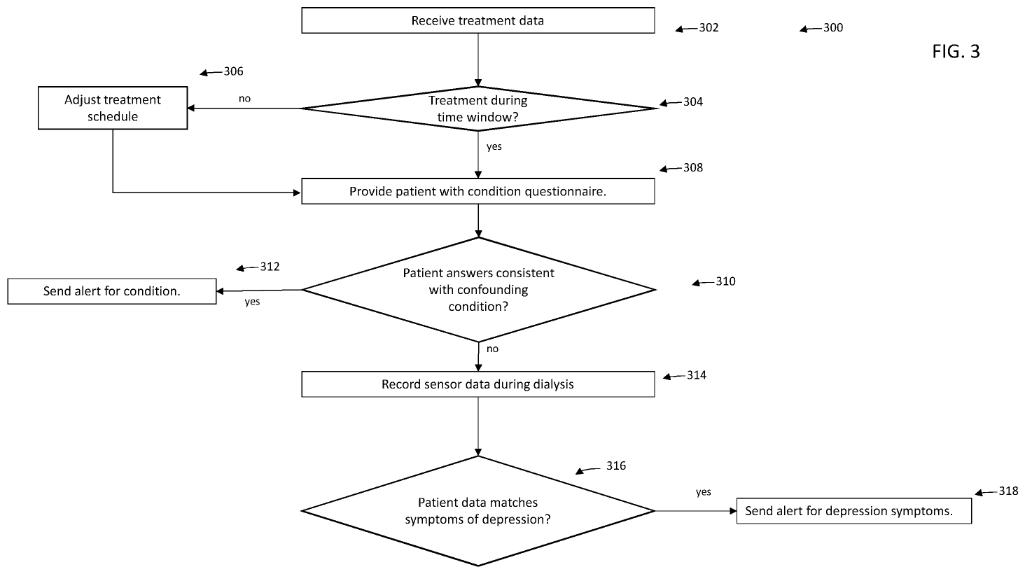

FIG. 3 illustrates an exemplary process 300 for identifying depression symptoms during a dialysis treatment. At step 302, the home dialysis unit receives treatment data representing one or more dialysis treatments to be carried out on a patient. The treatment data may include fluid volumes, timing, restrictions on temperature and/or pressure, and any other parameters used to properly set and control the dialysis cycler. For example, processor 202, in executing instructions 218 stored in memory 204, can receive information elements (e.g., from care coordination system 105, or the like) including indications a dialysis treatment to be carried out by the home dialysis unit 110.

At step 304, the timing of the scheduled treatment is compared to determine if treatment will correspond to a time window in which data is to be collected. The processor 202 may compare data 220 from data storage 206 and instructions 218 from memory 204 that include the preferred time windows for data collection with the projected schedule from the scheduling module 214. The scheduling module 214 will make an adjustment to the treatment schedule (step 306) if the timing does not match. For example, heart rate data may be collected between 2 am and 4 am, and then again between 4 am and 6 am. The scheduling module 214 can shift the scheduled treatment to begin at 1 am so that the treatment is ongoing during this window.

In some implementations, in which the processor 202 determines from the data 220 and instructions 218 that phase of treatment would be preferred for taking data, the scheduling module 214 may adjust so that the treatment phase is most likely to occur during that time window or windows. For example, where cyclic peritoneal dialysis is used and data collection is from 2 am to 4 am and from 4 am to 6 am, the treatment may be adjusted so that a drain/fill phase occurs around 4 am, thus allowing as much of the data-collection windows as possible to be uninterrupted during dwell phases.

At step 308, where other conditions may act as confounding factors with data for depression symptoms, the home dialysis unit 110 presents a questionnaire to the patient to identify these conditions. For example, questions may ask the patient to identify their energy level or ability to focus, to screen for symptoms of fatigue. In some implementations, the questionnaire may be stored in local data storage 220 and may be accessed by the user interface module 216 and presented to the patient, or to a caregiver for the patient, by means of the display 114 on the home dialysis unit 110. In some implementations, the questionnaire may first be accessed remotely via the communication module 208 and then presented locally to the user. A website or application may, in other embodiments, provide the questionnaire to a computer or mobile device accessible to the user. Alternatively, the questionnaire may be provided to a medical professional or other agent to communicate to the patient or caregiver, either in person or remotely by a phone conversation or some other means. Where the questionnaire is provided by the home dialysis unit 110, the answers may be recorded and stored within data storage 206 as part of a patient's profile and may be communicated remotely to the care coordination system 105. In other embodiments, step 308 may be optional, or selectively disabled or skipped, for example by the caregiver for the patient or other medical professional.

If a patient's answers to the questionnaire are symptomatic for a confounding condition such as fatigue (step 310), then an alert may be sent for the fatigue condition (step 312). The alert may be provided via the display of the home dialysis unit and may additionally be a communication to the care coordination system and to a remote medical professional administrating the patient's treatment. In some implementations, dialysis treatment may be temporarily halted or modified based on the confounding condition. For example, if the processor 202 identifies the questionnaire answers as consistent with severe fatigue, the cycler control 210 may be instructed not to start dialysis treatment or to reduce the number or duration of cycles conducted in the treatment. Similarly, instructions may be sent to the scheduling module 214 to cancel or delay upcoming scheduled treatment or to limit the frequency or duration of treatments.

In some cases, the dialysis treatment may continue as scheduled, but the processor 202 may adjust the collection of sensor data or how it is used. For example, if severe fatigue is identified, it may not be possible to identify the recorded data as depression symptoms due to the confounding condition. The processor 202 may therefore instruct the sensor control module 212 not to record heartbeats during the previously identified time window, or the processor may receive the sensor data 220 and record it in data storage 220 but may not evaluate the data for depression symptoms. Alternatively, the range of values that translate to depression symptoms may be altered based on the confounding condition. Certain sensor readings that might have been considered depression symptoms otherwise may instead be identified by the processor 202 as consistent with fatigue.

At step 314, if no confounding condition was found or if the identified confounding condition still allows for treatment and data collection, the sensor control module 212 controls the sensors 122 to collect sensor data during dialysis. Any appropriate sensor data may be collected, including electrical, optical, sonic, and/or movement data, among others known in the art. The data 220 may be stored in data storage 206 and may be analyzed by the processor 202 to identify a patient's heart rate along with other patient vitals. Where sensors are deployed to measure fluid outside the patient, the sensor data 220 may still be analyzed to determine a patient condition or identify patient symptoms.

At step 316, patient data is analyzed to determine if it matches symptoms of depression. This analysis may occur during patient treatment or after treatment is concluded. The controller 112 may, by means of the processor 202 and memory 202, analyze the collected patient data 220 stored in data storage 206 within the home dialysis unit 110. A care coordination system 105 or other connected medical system may analyze the data transmitted from the home dialysis unit 110 by means of the communication module 208. For example, heart rate data during certain portions of the patient's sleep cycle may be analyzed to see if the data matches a biomarker noted as correlated to depression. A recent study by Singapore's Nanyang Technological University has shown that heart rate sensors can be used to screen people for early signs of depression, the research is described in a paper recently published, Rykov Y, Thach T Q, Bojic I, Christopoulos G, Car J. Digital Biomarkers for Depression Screening With Wearable Devices: Cross-sectional Study With Machine Learning Modeling. JMIR Mhealth Uhealth. 2021 Oct. 25; 9(10):e24872. doi: 10.2196/24872. PMID: 34694233; PMCID: PMC8576601, the contents of which are incorporated by reference in their entirety.

The biomarker may be stored within the data storage 206 or may be accessed by means of the communication module 208. If the local processor 202 or remote system matches the patient data to a pattern correlated with depression, then an alert may be sent (step 318). The alert may be provided locally via the user interface module 216 and the display 114. An alert may also be communicated by the care coordination system 105 to a medical professional. This may prompt further screening for depression or modifications to the patient's treatment. In some implementations, identifying depression symptoms may cause the processor 202 to instruct the cycler control module 210 and scheduling module 214 to halt or delay further treatment until further actions is taken by a medical professional. In other implementations, modification to dialysis instructions may arrive remotely through the communication module 208 and may then be carried out by controller 112 via the processor 202 and memory 204.

FIGS. 4A-4B show an example of a peritoneal dialysis (PD) system 401, which is configured in accordance with an exemplary embodiment of the system described herein. In some implementations, the PD system 401 may be a home PD system, e.g., a PD system configured for use at a patient's home. The dialysis system 401 may include a dialysis machine 400 (e.g., a peritoneal dialysis machine 400, also referred to as a PD cycler) and in some embodiments the machine may be seated on a cart 434.

The dialysis machine 400 may include a housing 406, a door 408, and a cartridge interface including pump heads 442, 444 for contacting a disposable cassette, or cartridge 415, where the cartridge 415 is located within a compartment formed between the cartridge interface and the closed door 408 (e.g., cavity 420). Fluid lines 425 may be coupled to the cartridge 415 in a known manner, such as via a connector, and may further include valves for controlling fluid flow to and from fluid bags including fresh dialysate and warming fluid. In another embodiment, at least a portion of the fluid lines 425 may be integral to the cartridge 415. Prior to operation, a user may open the door 408 to insert a fresh cartridge 415, and to remove the used cartridge 415 after operation.

The cartridge 415 may be placed in the cavity 420 of the machine 400 for operation. During operation, dialysate fluid may be flowed into a patient's abdomen via the cartridge 415, and spent dialysate, waste, and/or excess fluid may be removed from the patient's abdomen via the cartridge 415. The door 408 may be securely closed to the machine 400. Peritoneal dialysis for a patient may include a total treatment of approximately 10 to 30 liters of fluid, where approximately 2 liters of dialysate fluid are pumped into a patient's abdomen, held for a period, e.g., about an hour, and then pumped out of the patient. This is repeated until the full treatment volume is achieved, and usually occurs overnight while a patient sleeps.

A heater tray 416 may be positioned on top of the housing 406. The heater tray 416 may be any size and shape to accommodate a bag of dialysate (e.g., a 5 L bag of dialysate) for batch heating. The dialysis machine 400 may also include a user interface such as a touch screen 418 and control panel 420 operable by a user (e.g., a caregiver or a patient) to allow, for example, set up, initiation, and/or termination of a dialysis treatment. In some embodiments, the heater tray 416 may include a heating element 435, for heating the dialysate prior to delivery into the patient.

Dialysate bags 422 may be suspended from hooks on the sides of the cart 434, and a heater bag 424 may be positioned in the heater tray 416. Hanging the dialysate bags 422 may improve air management as air content may be disposed by gravity to a top portion of the dialysate bag 422. Although four dialysate bags 422 are illustrated in FIG. 4B, any number “n” of dialysate bags may be connectable to the dialysis machine 400 (e.g., 1 to 5 bags, or more), and reference made to first and second bags is not limiting to the total number of bags used in a dialysis system 401. In some embodiments, connectors and tubing ports may connect the dialysate bags 422 and lines for transferring dialysate. Dialysate from the dialysate bags 422 may be transferred to the heater bag 424 in batches. For example, a batch of dialysate may be transferred from the dialysate bags 422 to the heater bag 424, where the dialysate is heated by the heating element 435. When the batch of dialysate has reached a predetermined temperature (e.g., approximately 98°−100° F., 37° C.), the batch of dialysate may be flowed into the patient. The dialysate bags 422 and the heater bag 424 may be connected to the cartridge 415 via dialysate bag lines or tubing 425 and a heater bag line or tubing 428, respectively. The dialysate bag lines 425 may be used to pass dialysate from dialysate bags 422 to the cartridge during use, and the heater bag line 428 may be used to pass dialysate back and forth between the cartridge and the heater bag 424 during use. In addition, a patient line 436 and a drain line 432 may be connected to the cartridge 415. The patient line 436 may be connected to a patient's abdomen via a catheter and may be used to pass dialysate back and forth between the cartridge and the patient's peritoneal cavity by the pump heads 442, 444 during use. The drain line 432 may be connected to a drain or drain receptacle and may be used to pass dialysate from the cartridge to the drain or drain receptacle during use.

Although in some embodiments, dialysate may be batch heated as described above, in other embodiments, dialysis machines may heat dialysate by in-line heating, e.g., continuously flowing dialysate through a warmer pouch positioned between heating elements prior to delivery into a patient. For example, instead of a heater bag for batch heating being positioned on a heater tray, one or more heating elements may be disposed internal to the dialysis machine. A warmer pouch may be insertable into the dialysis machine via an opening. It is also understood that the warmer pouch may be connectable to the dialysis machine via tubing (e.g., tubing 425), or fluid lines, via a cartridge. The tubing may be connectable so that dialysate may flow from the dialysate bags, through the warmer pouch for heating, and to the patient.

The touch screen 418 and the control panel 420 may allow an operator to input various treatment parameters to the dialysis machine 400 and to otherwise control the dialysis machine 400. In addition, the touch screen 418 may serve as a display. The touch screen 418 may function to provide information to the patient and the operator of the dialysis system 401. For example, the touch screen 418 may display information related to a dialysis treatment to be applied to the patient, including information related to a prescription.

The dialysis machine 400 may include a processing module 402 that resides inside the dialysis machine 400, the processing module 402 being configured to communicate with the touch screen 418 and the control panel 420. The processing module 402 may be configured to receive data from the touch screen 418 the control panel 420 and sensors, e.g., weight, air, flow, temperature, and/or pressure sensors, and control the dialysis machine 400 based on the received data. For example, the processing module 402 may adjust the operating parameters of the dialysis machine 400.

The dialysis machine 400 may be configured to connect to a network 403. The connection to network 403 may be via a wired and/or wireless connection. The dialysis machine 400 may include a connection component 404 configured to facilitate the connection to the network 403. The connection component 404 may be a transceiver for wireless connections and/or other signal processor for processing signals transmitted and received over a wired connection. Other medical devices (e.g., other dialysis machines) or components may be configured to connect to the network 403 and communicate with the dialysis machine 400.

The user interface portion such as the touch screen 418 and/or display 420 may include one or more buttons for selecting and/or entering user information. The touch screen 418 and/or display 420 may be operatively connected to a controller (not shown) and disposed in the machine 400 for receiving and processing the inputs to operate the dialysis machine 400.

Although FIGS. 4A-4B illustrate an exemplary embodiment of a dialysis machine in the form of a peritoneal dialysis system, a dialysis machine as referenced herein may also include a hemodialysis machine including HD machines designed for in-center use or at home use (e.g., home hemodialysis machine).

Some embodiments of the disclosed systems may be implemented, for example, using a storage medium, a computer-readable medium or an article of manufacture which may store an instruction or a set of instructions that, if executed by a machine (i.e., processor or microcontroller), may cause the machine to perform a method and/or operations in accordance with embodiments of the disclosure. In addition, a server or database server may include machine readable media configured to store machine executable program instructions. Such a machine may include, for example, any suitable processing platform, computing platform, computing device, processing device, computing system, processing system, computer, processor, or the like, and may be implemented using any suitable combination of hardware, software, firmware, or a combination thereof and utilized in systems, subsystems, components, or sub-components thereof. The computer-readable medium or article may include, for example, any suitable type of memory unit, memory device, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

To the extent used in this description and in the claims, a recitation in the general form of “at least one of [a] and [b]” should be construed as disjunctive. For example, a recitation of “at least one of [a], [b], and [c]” would include [a] alone, [b] alone, [c] alone, or any combination of [a], [b], and [c].

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A system for detecting symptoms of depression in a home dialysis patient, the system comprising:
a home dialysis system configured to:
carry out a home dialysis treatment on a patient;
during the home dialysis treatment, detect one or more symptoms of depression; and
generate a medical alert based on the detected one or more symptoms.

2. The system of claim 1,
wherein a symptom of the one or more symptoms of depression is a sleep behavior; and
wherein the home dialysis treatment occurs at least partially while the patient is asleep.

3. The system of claim 2, wherein the home dialysis system is arranged and configured to:
identify a time window associated with the sleep behavior; and
schedule the home dialysis treatment to occur during the identified time window.

4. The system of claim 3,
wherein the home dialysis treatment is a peritoneal dialysis treatment including a dwell phase; and
wherein the home dialysis treatment is scheduled so that the dwell phase occurs during the identified treatment window.

5. The system of claim 1, further comprising a medical sensor integrated within the home dialysis system that provides data to detect the one or more depression symptoms.

6. The system of claim 5, wherein the medical sensor is a pulse oximeter.

7. The system of claim 5, wherein the medical sensor is an electrocardiogram.

8. The system of claim 5, wherein the medical sensor is a photoplethysmography device or an optical heart rate monitor.

9. The system of claim 5, wherein the medical sensor is an ultrasonic sensor.

10. A method for detecting symptoms of depression during home dialysis, comprising:
carrying out a home dialysis treatment on a patient;
during the home dialysis treatment, detecting one or more symptoms of depression; and
generating a medical alert based on the detected one or more symptoms.

11. The method of claim 10,
wherein a symptom of the one or more symptoms of depression is a sleep behavior; and
wherein the home dialysis treatment occurs at least partially while the patient is asleep.

12. The method of claim 10, further comprising:
identifying a time window associated with the sleep behavior; and
scheduling the home dialysis treatment to occur during the identified time window.

13. The method of claim 12,
wherein the home dialysis treatment is a peritoneal dialysis treatment including a dwell phase; and
wherein the home dialysis treatment is scheduled so that the dwell phase occurs during the identified treatment window.

14. The method of claim 10, wherein the data is detected by a medical sensor integrated within the home dialysis system.

15. The method of claim 14, wherein the medical sensor is a pulse oximeter.

16. The method of claim 14, wherein the medical sensor is an electrocardiogram.

17. The method of claim 14, wherein the medical sensor is a photoplethysmography device or an optical heart rate monitor.

18. The method of claim 14, wherein the medical sensor is an ultrasonic sensor.

19. A system for detecting symptoms of depression during home dialysis, comprising:
a home dialysis apparatus; and
a controller for the home dialysis apparatus, the controller comprising processing circuitry and a memory storage device coupled to the processing circuitry, the memory storage device comprising instructions executable by the processing circuitry, which when executed cause the controller to:
send a control signal to the home dialysis apparatus comprising an indication to initiate a home dialysis treatment on a patient;
during, the home dialysis treatment, detect one or more symptoms of depression; and
generate a medical alert based on the detected one or more symptoms.

20. The system of claim 19, wherein a symptom of the one or more symptoms of depression is a sleep behavior and wherein the home dialysis treatment occurs at least partially while the patient is asleep.
